(19) United States
(12) Patent Application Publication
(10) Pub. No.: US 2021/0063585 A1
(43) Pub. Date: Mar. 4, 2021

(54) ELECTRONIC CASSETTE

(71) Applicant: FUJIFILM Corporation, Tokyo (JP)

(72) Inventors: Shinsuke NOGUCHI, Kanagawa (JP); Hirotoshi ONO, Kanagawa (JP); Naoyuki NISHINO, Kanagawa (JP); Takeyasu KOBAYASHI, Kanagawa (JP)

(73) Assignee: FUJIFILM Corporation, Tokyo (JP)

(21) Appl. No.: 17/002,293

(22) Filed: Aug. 25, 2020

(30) Foreign Application Priority Data
Sep. 3, 2019 (JP) 2019-160654

(51) Int. Cl. G01T 1/17; A61B 6/00

(52) U.S. Cl. CPC G01T 1/17; A61B 6/4283

(57) ABSTRACT

An electronic cassette includes an image detection unit that detects a radiographic image of a subject, a housing that houses the image detection unit and has a front surface, a rear surface, and side surfaces, a first indicator that is provided on the front surface and outside an imaging region of the image detection unit, and emits light to indicate a center of the imaging region, and a second indicator that is provided on the front surface and outside the imaging region, and emits light to represent a possibility of detection of the radiographic image.

### CROSS-REFERENCE TO RELATED APPLICATIONS

This application claims priority under 35 U.S.C § 119(a) to Japanese Patent Application No. 2019-160654 filed on 3 Sep. 2019. The above application is hereby expressly incorporated by reference, in its entirety, into the present application.

### BACKGROUND OF THE INVENTION

### 1. Field of the Invention

The present invention relates to an electronic cassette that images a subject using radiation, such as X-rays.

### 2. Description of the Related Art

An electronic cassette has been widely used in medical radiography. For example, in X-ray imaging, an electronic cassette is a portable X-ray image detection device having an image detection unit that detects an X-ray image of a subject by receiving X-rays transmitted through the subject.

There is a case where the electronic cassette has an indicator indicating a state of the electronic cassette, such as whether or not a state in which imaging using radiation is possible is brought. As the indicator, an indicator that is provided on a side surface (JP2015-190925A (corresponding to US2015/276944A1)) or a rear surface of the electronic cassette is known.

In recent years, there is an example where an indicator that suggests a center of an imaging region is provided on a rear surface or a side surface of an electronic cassette, and is extended to a front surface of the electronic cassette (JP2016-063875A (corresponding to US2016/081638A1)). There is also an example where an indicator that indicates a “ready state” representing an imaging standby state or “data transmission” representing that image data is being transmitted is provided on a front surface of an electronic cassette (JP2014-195480A).

### SUMMARY OF THE INVENTION

In order to report a state of the electronic cassette, or the like to a radiological technician, there is demand for providing one or a plurality of indicators on a front surface that is a surface of the electronic cassette on a subject side. The reason is for enabling easy confirmation of the state of the electronic cassette, or the like in a state in which the electronic cassette is disposed for use in imaging.

However, in the electronic cassette of the related art, one kind of indicator can be confirmed from the front surface side of the electronic cassette. After all, another indicator on a side surface, a rear surface, or the like of the electronic cassette or display on a console as a control device should be confirmed in order to confirm a status other than a status indicated by the indicator, and imaging cannot be efficiently performed. For example, in the electronic cassette of the related art, any one of the indicator suggesting the center of the imaging region, the indicator indicating the ready state or data transmission, and the like is merely provided on the front surface of the electronic cassette.

There are a plurality of statuses that should be reported to the radiological technician by the electronic cassette. However, it is hard to provide all indicators representing various statuses to be reported on the front surface of the electronic cassette while the specific shape and size of the electronic cassette are maintained. On the other hand, statuses that should be confirmed from the front surface side of the electronic cassette during imaging in order to efficiently advance imaging are at least a position of the center of the imaging region and a possibility of detection of a radiographic image.

Accordingly, an object of the invention is to provide an electronic cassette that enables confirmation a center of an imaging region and a possibility of detection of a radiographic image from a front surface side.

The invention provides an electronic cassette comprising an image detection unit that detects a radiographic image of a subject using radiation transmitted through the subject, a housing that houses the image detection unit and has a front surface directing toward the subject in a case of detecting the radiographic image, a rear surface facing the front surface, and side surfaces connecting the front surface and the rear surface, a first indicator that is provided on the front surface and outside an imaging region of the image detection unit, and emits light to indicate a center of the imaging region, and a second indicator that is provided on the front surface and outside the imaging region, and emits light to represent a possibility of detection of the radiographic image.

It is preferable that the first indicator and the second indicator are provided at a plurality of places of the housing.

It is preferable that the first indicator and the second indicator are provided on each of four sides of the front surface.

It is preferable that the second indicator is provided in a connection portion of the front surface and each of the side surfaces.

It is preferable that a display color of the first indicator and a display color of the second indicator are different.

It is preferable that the display color of the second indicator is green.

It is preferable that, in a case where another electronic cassette having the first indicator is usable, the first indicator has a variable display color and emits light in a color different from the first indicator of the other electronic cassette.

It is preferable that, in a case where the second indicator and the first indicator are turned on, the second indicator is turned on brighter than the first indicator.

It is preferable that a display area of the second indicator on the front surface is greater than a display area of the first indicator on the front surface.

It is preferable that the first indicator has a long shape in a direction perpendicular to a side of the front surface, and the second indicator has a long shape in a direction parallel to the side of the front surface.

It is preferable that the first indicator has a shape extended to each of the side surfaces of the housing.

It is preferable that a substrate that controls the first indicator and a substrate that controls the second indicator are different from a control substrate of the image detection unit.

It is preferable that the electronic cassette further comprises a first light shielding member that shields the image detection unit from the substrate that controls the first indicator and the substrate that controls the second indicator.

It is preferable that the electronic cassette further comprises a second light shielding member that is provided between the first indicator and the second indicator to shield the first indicator and the second indicator from each other.

It is preferable that the electronic cassette further comprises a third indicator that is provided on the front surface of the housing and outside the imaging region, and emits light to represent an orientation of the image detection unit.

It is preferable that the third indicator is provided on each of four sides of the front surface and is alternatively turned on.

The electronic cassette of the invention enables confirmation of the center of the imaging region and the possibility of detection of the radiographic image from a front surface side.

### BRIEF DESCRIPTION OF THE DRAWINGS

FIG. 1 is a perspective view of an electronic cassette.

FIG. 2 is a plan view showing a front surface of the electronic cassette.

FIG. 3 is a plan view showing a right side surface of the electronic cassette.

FIG. 4 is a plan view showing a rear surface of the electronic cassette.

FIG. 5 is an explanatory view showing each indicator of a display unit.

FIG. 6 is an explanatory view showing an area of each of indicators on the front surface.

FIG. 7 is an XZ sectional view of a second indicator portion showing the internal configuration of the electronic cassette.

FIG. 8 is a plan view showing a state in which a front cover is detached.

FIG. 9 is an explanatory view showing light leakage.

FIG. 10 is an explanatory view showing a modification example of a display unit.

FIG. 11 is an explanatory view showing light emission states of a first indicator and a second indicator in the modification example.

FIG. 12 is a plan view showing a right side surface of a modification example.

FIG. 13 is a plan view showing a rear surface of the modification example.

FIG. 14 is a plan view of a front surface showing a display unit of a modification example.

FIG. 15 is a plan view of a side surface showing the display unit of the modification example.

FIG. 16 is a plan view of a display unit in which arrangement of indicators is changed.

### DESCRIPTION OF THE PREFERRED EMBODIMENTS

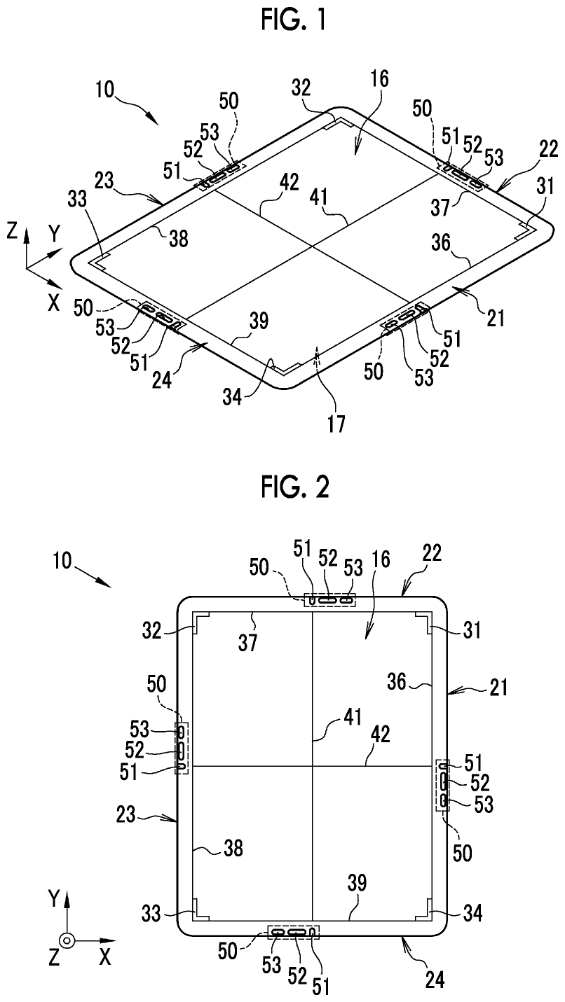

As shown in FIGS. 1 to 4, an electronic cassette 10 has a front surface 16, a rear surface 17 facing the front surface 16, and side surfaces 21 to 24 connecting the front surface 16 and the rear surface 17, and has, for example, a rectangular parallelepiped shape as a whole. The electronic cassette 10 has, for example, a shape and a size conforming to the Japanese Industrial Standards (JIS) standard (JIS Z4905) or the International Organization for Standardization (ISO) standard (ISO 4090:2001).

In the following description, a direction parallel to the side surface 22 and the side surface 24 is the X direction, a direction parallel to the side surface 21 and the side surface 23 is the Y direction, and a direction of the front surface 16 viewed from the rear surface 17 is the Z direction. In a case where the electronic cassette 10 is viewed from the front surface 16 or the rear surface 17, a side formed by the side surface 21 is referred to as a right side of the electronic cassette 10, a side formed by the side surface 22 is referred to as an upper side of the electronic cassette 10, a side formed by the side surface 23 is referred to as a left side of the electronic cassette 10, and a side formed by the side surface 23 is referred to as a lower side. The front surface 16 is a surface visible in a case where the electronic cassette 10 is viewed in a front view from a Z-direction positive side, and is a front surface of the electronic cassette 10 on which radiation is incident. The rear surface 17 is a surface visible in a case where the electronic cassette 10 is viewed in a front view from a Z-direction negative side, and is a rear surface of the electronic cassette 10. The side surface 21 is a surface visible in a case where the electronic cassette 10 is viewed in a front view from an X-direction positive side, and is a right side surface of the electronic cassette 10. The side surface 22 is a surface visible in a case where the electronic cassette 10 is viewed in a front view from a Y-direction positive side, and is an upper side surface of the electronic cassette 10. The side surface 23 is a surface visible in a case where the electronic cassette 10 is viewed in a front view from an X-direction negative side, and is a left side surface of the electronic cassette 10. The side surface 24 is a surface visible in a case where the electronic cassette 10 is viewed in a front view from a Y-direction negative side, and is a lower side surface of the electronic cassette 10.

The front surface 16 is directed toward a subject (not shown) in a case where a radiographic image is detected, that is, in a case where imaging is performed. For this reason, in a case where imaging is performed, the subject or the like comes into contact with the front surface 16. As a result, there is a case where a part or the whole of the front surface 16 is hidden by the subject or the like. Note that, excluding a case where the electronic cassette 10 is set in a predetermined holder positioned in advance, and the position, orientation, and the like of the electronic cassette 10 can be confirmed by the holder, it is rare that the whole of the electronic cassette 10 is completely hidden by the subject or the like in at least a preparation stage of imaging in which the arrangement of the subject and the electronic cassette 10 is adjusted. For this reason, in most cases, at least one side of the right side, the left side, the upper side, or the lower side of the electronic cassette 10 is visible from the front surface 16 side during imaging (at least the preparation stage of imaging).

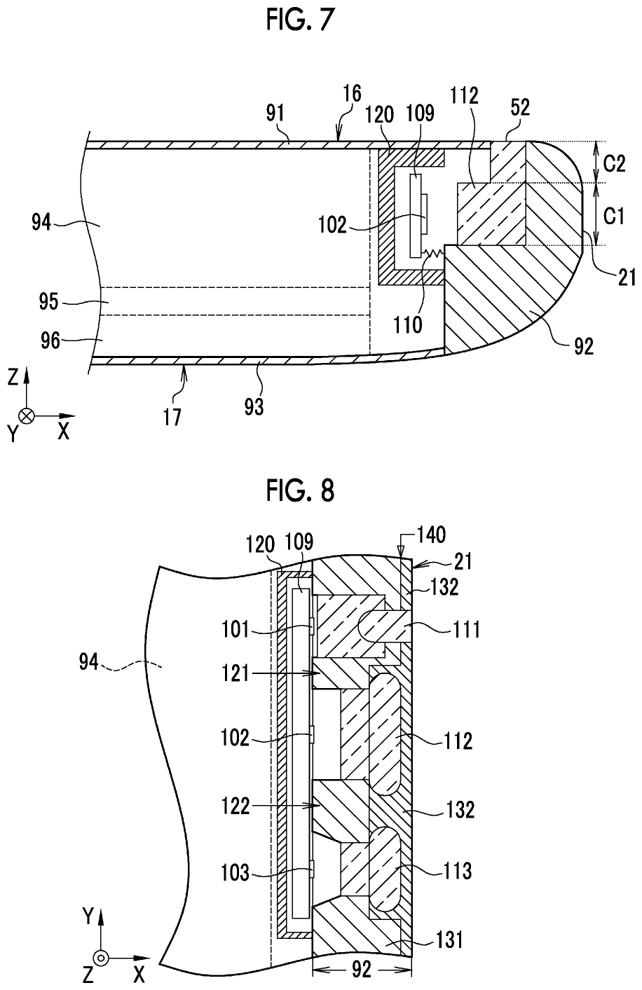

The front surface 16 is substantially a plane. The front surface 16 shows an imaging region by markers 31 to 34 and lines 36 to 39 connecting the markers 31 to 34. The imaging region refers to a region (so-called effective pixel region) where there are pixels contributing to a radiographic image in an image detection unit 94 (see FIG. 7). The markers 31 to 34 represent corners of the imaging region, and the lines 36 to 39 connecting the markers 31 to 34 represent ends of the imaging region. An intersection point of crosslines 41 and 42 represents the center of the imaging region.

The electronic cassette 10 has a display unit 50 on the front surface 16. In the embodiment, the display unit 50 comprises a first indicator 51, a second indicator 52, and a third indicator 53. The display unit 50 including the first indicator 51, the second indicator 52, and the third indicator 53 is provided at a plurality of places of a housing of the electronic cassette 10. Specifically, one display unit 50 including the first indicator 51, the second indicator 52, and the third indicator 53 is provided on each of four sides of the front surface 16, that is, the upper, lower, right, and left sides of the electronic cassette 10. The reason is for enabling confirmation of the first indicator 51, the second indicator 52, and the third indicator 53 of the display unit 50 on one to three sides of the upper, lower, right, or left side of the electronic cassette 10 from the front surface 16 side even though one to three other sides are covered with the subject or the like.

The first indicator 51 is provided outside the imaging region (a region indicated by the lines 36 to 39) of the image detection unit 94, and emits light to show the center of the imaging region to the radiological technician who views the electronic cassette 10 from the front surface 16. The term “indicate the center of the imaging region” refers to suggest at least one of a position or a direction where the center of the imaging region is present. For example, the first indicator 51 in of the display unit 50 on each of the right side and the left side is on the extension of the crossline 42. For this reason, the first indicator 51 in the display unit 50 on each of the right side and the left side suggests a center position of the imaging region in the Y direction with the position of the first indicator 51. The first indicator 51 in the display unit 50 on each of the right side and the left side also suggests a direction, in which the center of the imaging region is present, with the shape of the first indicator 51. On the other hand, the first indicator 51 in the display unit 50 on each of the upper side and the lower side is on the extension of the crossline 41. For this reason, the first indicator 51 in the display unit 50 on each of the upper side and the lower side suggests a center position of the imaging region in the X direction with the position of the first indicator 51. The first indicator 51 in the display unit 50 on each of the upper side and the lower side also suggests a direction, in which the center of the imaging region is present, with the shape of the first indicator 51.

In a case where another electronic cassette having the first indicator 51 is usable along with the electronic cassette 10, the first indicator 51 has a variable display color, and emits light in a color different from the first indicator 51 of the other electronic cassette. The other electronic cassette having the first indicator 51 is another electronic cassette having the same configuration as the electronic cassette 10 or an electronic cassette that is an electronic cassette 10 having a different size or an electronic cassette of a related model, such as a successor, and has the first indicator 51 (including another indicator that functions in the same manner as the first indicator). That is, the color (hereinafter, referred to as a display color) displayed when the first indicator 51 emits light is an identification color for identifying the electronic cassette 10. Specifically, the first indicator 51 emits light in one specific display color selected from a plurality of colors of cyan, magenta, orange, green, and violet by the radiological technician or a control device (so-called console) of a system.

In a radiography system (not shown) using the electronic cassette 10, usually, a plurality of electronic cassettes 10 different in size or the like are provided, and one of a plurality of electronic cassettes 10 is selected and used in imaging. A plurality of electronic cassettes 10 are, for example, an electronic cassette for free imaging, an electronic cassette for a decubitus imaging table, an electronic cassette for an upright imaging table, and the like. An error in selection of the electronic cassette 10 for use in imaging from a plurality of electronic cassettes 10 results in degradation of a workflow. There is also a need to discriminate the electronic cassette 10 that is connected to the control device (so-called console (not shown)) of the radiography system. For this reason, the identification color of the electronic cassette 10 for use in imaging is indicated on the control device, and the electronic cassette 10 for use in imaging turns on the first indicator 51 in the identification color. In this way, the first indicator 51 emits light in a specific identification color, whereby the radiological technician not only can know the center of the imaging region, but also can visually know whether or not the electronic cassette 10 is to be used in imaging with the display of the first indicator 51. In a case where a plurality of electronic cassettes 10 are usable, it is possible to restrain a problem that imaging is performed using an improper electronic cassette 10, and reimaging is unavoidable.

All of the first indicators 51 of the display units 50 on the respective sides are turned on and off simultaneously. All of the display colors are also the same. This is for identifying the electronic cassette 10 and for indicating the center of the imaging region by any one of a plurality of first indicators 51.

The second indicator 52 is provided outside the imaging region and emits light to represents a possibility of detection of a radiographic image. The term “the possibility of detection of the radiographic image” refers to a status indicating whether or not the electronic cassette 10 is ready to detect the radiographic image. That is, the second indicator 52 represents a so-called ready state (a state in which radiation can be exposed). The second indicator 52 is turned on to represent the ready state and is turned off to represent an unready state. The reason that the second indicator 52 reports the possibility of detection of the radiographic image is because the radiological technician needs to clearly know a state of the possibility of detection of the radiographic image due to a wasteful increase in exposure dose to the subject in a case where imaging is performed in a state in which radiation can be exposed.

In the embodiment, a display color of the second indicator 52 is set to an invariable intrinsic color. This is to restrain the radiological technician from erroneously recognizing the ready state by erroneously recognizing turning-on of the first indicator 51 or the third indicator 53 as turning-on of the second indicator 52. Specifically, the display color of the second indicator 52 is green. This is because the display (green flag) of green is a color that is easy for the radiological technician to naturally recognize as the ready state.

The electronic cassette 10 sets the display colors of the first indicator 51 and the third indicator 53 to a display color different from the second indicator 52 in a case where the display color of at least one of the first indicator 51 or the third indicator 53 is variable. That is, the electronic cassette 10 prohibits the display colors of the first indicator 51 and the third indicator 53 from being set to the same color as the display color of the second indicator 52. In the embodiment, the display color of the first indicator 51 is variable, and the third indicator 53 is turned on in an invariable intrinsic color (for example, white) different from the second indicator 52. For this reason, the electronic cassette 10 sets the display color of the first indicator 51 to a display color different from at least the second indicator 52. As a result, in the electronic cassette 10, the display color of the first indicator 51 and the display color of the third indicator 53 are different from the display color of the second indicator. With this, the second indicator 52 can be recognized without being erroneously recognized as another indicator.

The second indicator 52 is provided in a connection portion of the front surface 16 and each of the side surfaces 21 to 24. That is, the second indicator 52 is provided near each side of the electronic cassette 10 where the display unit 50 is present. The reason is for allowing the second indicator 52 to be hardly hidden with the subject or the like and enabling more reliable confirmation of the second indicator 52 from the front surface 16 side even though the subject or the like is superimposed on the front surface 16. The configuration in which second indicator 52 is provided in the connection portion of the front surface 16 and each of the side surfaces 21 to 24 also contributes to maximizing the imaging region (enabling using the image detection unit 94 having a large imaging region) without causing an increase in shape and size of the appearance of the electronic cassette 10. This is because at least one of an internal space or a space on the front surface 16 needed for mounting the second indicator 52 can be suppressed. Accordingly, the configuration in which the second indicator 52 is provided in the connection portion of the front surface 16 and each of the side surfaces 21 to 24 is particularly useful in the electronic cassette 10 in which the shape and size are limited to specific shape and size to conform to a specific standard, such as the JIS standard or the ISO standard, as in the embodiment.

All of the second indicators 52 in the display units 50 on the respective sides are turned on and off simultaneously. The display colors are also the same. The reason is for allowing the possibility of detection of the radiographic image to be known by any one of a plurality of second indicators 52.

In a case where the second indicator 52 and the first indicator 51 are turned on, the second indicator 52 is turned on brighter than the first indicator 51. For example, in a case where the second indicator 52 and the first indicator 51 are turned on, the luminance of the second indicator 52 is greater than the luminance of the first indicator 51. In a case where the third indicator 53 is turned on, the second indicator 52 is displayed brighter than the first indicator 51 and the third indicator 53. That is, the second indicator 52 is turned on the brightest among a plurality of indicators constituting the display unit 50. The reason is for enabling correct and reliable recognition of the possibility of detection of the radiographic image by displaying the second indicator 52 relatively intensely with respect to the first indicator 51 and the third indicator 53 even though the first indicator 51 and the third indicator 53 are lined with the second indicator 52.

The third indicator 53 is provided outside the imaging region and emits light to represent an orientation of the image detection unit 94. The term “the orientation of the image detection unit 94” is substantially synonymous with an orientation of a radiographic image before rotation processing is executed. For this reason, the radiological technician performs imaging while adjusting the arrangement of the electronic cassette 10 according to the display of the third indicator 53, and can obtain a radiographic image that does not need the rotation processing after imaging. Similarly to the second indicator 52, the third indicator 53 is provided in the connection portion of the front surface 16 and each of the side surfaces 21 to 24. This is because the imaging region is maximized by suppressing at least one of an internal space or a space on the front surface 16 needed for mounting the third indicator 53 without causing an increase in shape and size of the appearance of the electronic cassette 10. The configuration in which the third indicator 53 is provided in the connection portion of the front surface 16 and each of the side surfaces 21 to 24 is particularly useful in a case where the shape and size of the electronic cassette 10 conform to a specific standard.

All of the four display units 50 on the respective sides of the electronic cassette 10 include the third indicator 53. That is, the third indicator 53 is provided on each of the four sides of the front surface 16. Note that one of the third indicators 53 is alternatively turned on. This is for reporting vertical and horizontal orientations of the image detection unit 94 with turning-on of any one of a plurality of third indicators 53. In the embodiment, a direction that becomes the “top” of the radiographic image in a state in which the rotation processing is executed is indicated with turning-on of the third indicator 53.

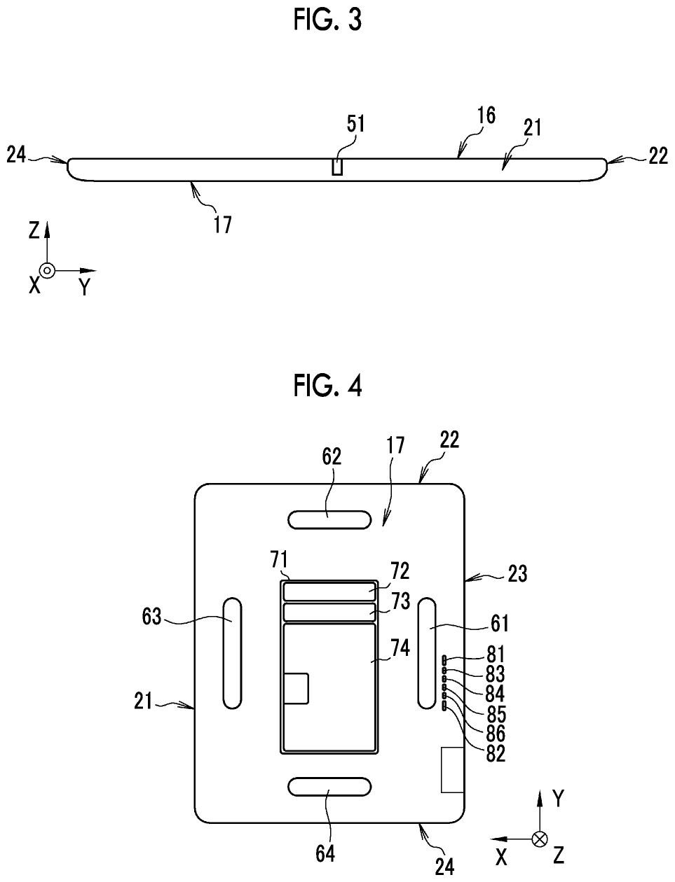

As shown in FIG. 3, the first indicator 51 constituting the display unit 50 on the right side has a shape extended to the side surface 21 as the right side surface of the electronic cassette 10. Similarly, the first indicator 51 constituting the display units 50 on the upper side, the left side, and the lower side have shapes extended to the side surface 22, the side surface 23, and the side surface 24, respectively (not shown). The reason is for identifying the electronic cassette 10 and allowing the center of the imaging region to be known on the side surfaces 21 to 24 of the electronic cassette 10. Since the electronic cassettes 10 are usually stored densely in an erected state in a cradle (not shown), as described above, the first indicators 51 have the shape extended to the respective side surfaces 21 to 24 of the electronic cassette 10, whereby the electronic cassette 10 for use in imaging can be instantly identified with the display colors of the first indicators 51 extended to the respective side surfaces 21 to 24.

As shown in FIG. 4, the electronic cassette 10 has anti-slipping rubber members 61 to 64 on the rear surface 17. With this, in a case where the electronic cassette 10 is disposed on a bed or the like, it is possible to restrain positional misalignment of the electronic cassette 10. The electronic cassette 10 integrally has a first rear display unit 72, a second rear display unit 73, and a battery pack 74 in a center portion 71 of the rear surface 17. The first rear display unit 72 displays a remaining quantity of the battery pack 74, an operation mode, and the like. The second rear display unit 73 displays a model number, a specification, other notes, and the like of the electronic cassette 10. The battery pack 74 supplies electric power to the electronic cassette 10. The battery pack 74 is mounted in an attachable and detachable manner and a replaceable manner, and can be reused through charging. Furthermore, operating units 81 and 82 and one or a plurality of rear indicators 83 to 86 are provided on the rear surface 17 of the electronic cassette 10. The operating units 81 and 82 are pressed simultaneously such that a state is brought in which the operation mode of the electronic cassette 10 is changeable. After the state is brought in which the operation mode is changeable, the operation mode can be selected by pressing the operating unit 81 or the operating unit 82 is pressed. The rear indicators 83 to 86 are provided between the operating unit 81 and the operating unit 82, and display statuses of the electronic cassette 10 not displayed on the front surface 16, respectively. The rear indicators 83 to 86 display, for example, an electrical conduction state (ON/OFF) of the electronic cassette 10, a communication state with the console or the like, other errors, and the like with turning-on, turning-off, colors during turning-on, blinking, or the like.

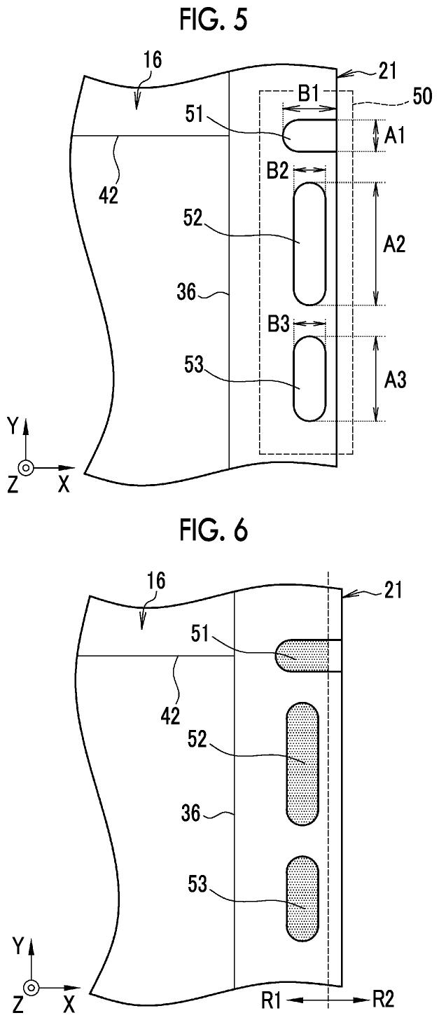

As shown in FIG. 5, the first indicator 51 has a long shape in a direction perpendicular to the side of the front surface 16, and the second indicator 52 has a long shape in a direction parallel to the side of the front surface 16. Similarly to the second indicator 52, the third indicator 53 has a long shape in a direction parallel to the side of the front surface 16. For example, lengths in the Y direction of the first indicator 51, the second indicator 52, and the third indicator 53 of the display unit 50 on the right side are “A1”, “A2”, and “A3, respectively, and lengths in the X direction of the first indicator 51, the second indicator 52, and the third indicator 53 are “B1”, “B2”, and “B3”, respectively. In this case, in regard to the first indicator 51, “A1<B1” is satisfied. The first indicator 51 is formed in a long shape in a direction perpendicular to the side (in FIG. 5, the right side of the front surface 16), on which the first indicator 51 is present, in this way, whereby it is possible to intuitively suggest a direction of the center of the imaging region.

In regard to the second indicator 52, “A2>B2” is satisfied. The second indicator 52 is formed in a long shape parallel to the side, on which second indicator 52 is present, in this way, whereby it is possible to allow the second indicator 52 to be hardly hidden by the subject or the like even though the subject or the like is superimposed on the front surface 16, and to enable more reliable confirmation of the second indicator 52 from the front surface 16 side.

In regard to the third indicator 53, “A3>B3” is satisfied. The third indicator 53 is formed in a long shape parallel to the side, on which the third indicator 53 is present, in this way, whereby it is possible to allow the third indicator 53 to be hardly hidden by the subject or the like even though the subject or the like is superimposed on the front surface 16, and to enable more reliable confirmation of the third indicator 53 from the front surface 16 side.

A length along the side of the second indicator 52 among the first indicator 51, the second indicator 52, and the third indicator 53 is relatively made the longest. That is, “A2>A1” and “A2>A3” are satisfied. In the embodiment, “A2>A3>A1” is satisfied. In this way, the length of the second indicator 52 is relatively made the longest, whereby the second indicator 52 is made the most conspicuous. As a result, even though the first indicator 51 and the third indicator 53 are lined with the second indicator 52, the radiological technician can accurately and reliably recognize the possibility of detection of the radiographic image.

In comparison of the display areas of the first indicator 51 and the second indicator 52 on the front surface 16, the display area of the second indicator 52 on the front surface 16 is greater than the display area of the first indicator 51 on the front surface 16. In comparison of the display areas including the third indicator 53 as well, the display area of the second indicator 52 is the greatest. That is, in a case where the display area of the first indicator 51 is “S1,” the display area of the second indicator 52 is “S2”, and the display area of the third indicator 53 is “S3” (all are not shown), at least “S2>S1” and “S2>S3” are satisfied. In this way, the display area of the second indicator 52 is made relatively large, whereby the second indicator 52 is made the most conspicuous. As a result, even though the first indicator 51 and the third indicator 53 are lined with the second indicator 52, the radiological technician can accurately and reliably recognize the possibility of detection of the radiographic image.

In comparison of the display areas of the first indicator 51, the second indicator 52, and the third indicator 53 as described above, as shown in FIG. 6, comparison may be performed in view of only a planar portion of the front surface 16. This is because a magnitude relationship of the display areas in the planar portion of the front surface 16 is dominant in relative visibility of each indicator in a case where the electronic cassette 10 is viewed from the front surface 16. Specifically, for example, on the right side of the front surface 16, in a case where a region R1 is a plane and a region R2 is curved by chamfering or the like, and in a case where the display areas (in FIG. 6, areas of hatched portions) of the first indicator 51, the second indicator 52, and the third indicator 53 within a range of the region R1 are σ1, σ2, and σ3, at least “σ2>σ1” and “σ2>σ3” are satisfied. Of course, it is preferable that “S2>S1” and “S2>S3”, and “σ2>σ1” and “σ2>σ3” are satisfied.

As shown in FIG. 7, the electronic cassette 10 has a front plate 91, a frame 92, and a rear plate 93. The front plate 91, the frame 92, and the rear plate 93 constitute a housing of the electronic cassette 10, and the image detection unit 94 is housed in the housing.

The front plate 91 is a member constituting the front surface 16 of the electronic cassette 10, and at least a portion corresponding to the image detection unit 94 is formed using a material, such as carbon, which transmits radiation. The frame 92 is a member constituting the side surfaces 21 to 24 of the electronic cassette 10, and for example, is formed using a lightweight metal, such as aluminum. The rear plate 93 is a member constituting the rear surface 17 of the electronic cassette 10, and for example, similarly to the frame 92, is formed using a lightweight metal, such as aluminum.

The image detection unit 94 detects a radiographic image of the subject using radiation transmitted through the subject. In the electronic cassette 10, as the image detection unit 94, either of a direct conversion type detector that directly converts radiation into an electric signal or an indirect conversion type detector that converts radiation into visible light and photoelectrically converts visible light can be used. In the embodiment, the image detection unit 94 is of an indirect conversion type. On a side the image detection unit 94 facing the rear surface 17, a control substrate 96 that controls the image detection unit 94 and other units of the electronic cassette 10 is provided through a radiation shielding member 95, such as a lead plate, which shields radiation. Examples of the control substrate 96 include a flexible substrate or the like.

Each indicator constituting the display unit 50 has a light emitter, a light guide that guides light emitted from the light emitter, and a substrate that controls light emission, a light emission amount, and the like of the light emitter to control the indicator. The light emitter is a semiconductor light emitting element, such as a light emitting diode (LED), a lamp using a heating wire or gas, or the like. In the embodiment, the light emitter is an LED. For this reason, for example, the second indicator 52 includes a second indicator LED 102 as a light emitter, a second light guide 112, and an LED substrate 109. The second indicator LED 102 is provided on the LED substrate 109 separate from the control substrate 96. The LED substrate 109 is a substrate that controls the second indicator 52. The control of the indicator refers to control of on or off of the indicator, a light emission amount, and the like. The LED substrate 109 controls the light emission, the light emission amount, and the like of the second indicator LED 102. As a result, the LED substrate 109 controls the second indicator 52. The LED substrate 109 is connected to, for example, the frame 92 by a fixing member 110, and is movable in any one direction or a plurality of directions among the respective directions of XYZ. The reason is for absorbing vibration or the like applied to the electronic cassette 10. The second indicator LED 102 emits green light. The second light guide 112 has a two-step configuration of a first portion C1 that receives light emitted from the second indicator LED 102 and guides received light, and a second portion C2 that is connected to the first portion C1 and exposes a part of a surface on the front surface 16.

The surface of the second portion C2 of the second light guide 112 exposed on the front surface 16 is flush with the front surface 16 in the embodiment. Note that the surface of the second portion C2 of the second light guide 112 exposed on the front surface 16 may be recessed with respect to the front surface 16. In a case where the surface of the second portion C2 of the second light guide 112 exposed on the front surface 16 is flush with the front surface 16 or is recessed with respect to the front surface 16, it is possible to reduce problems that the second light guide 112 is caught in the subject or the like during imaging, and the second light guide 112 is damaged, deformed, or the like due to getting caught in the subject or the like. The same applies to the first indicator 51 and the third indicator 53. The electronic cassette 10 further comprises a first light shielding member 120 that shields the LED substrate 109 with respect to the image detection unit 94.

As shown in FIG. 8, the first indicator 51 and the third indicator 53 have the same configuration as the second indicator 52. Specifically, the first indicator 51 includes a first indicator LED 101 and a first light guide 111. The first indicator LED 101 has a variable light emission color. For this reason, it is possible to change the display color of the first indicator 51 by changing the light emission color of the first indicator LED 101. The first light guide 111 is the same as the second light guide 112 in that a two-step configuration of a first portion that receives light emitted from the first indicator LED 101 and guides received light and a second portion that is connected to the first portion and exposes a part of a surface on the front surface 16 is made. Note that the first light guide 111 is composed conforming to the shape or the like of the first indicator 51, and for example, the second portion is formed in a shape protruding from the first portion in the X direction unlike the second light guide 112.

The third indicator 53 includes a third indicator LED 103 and a third light guide 113. The third indicator LED 103 emits light in a specific color, for example, white in the embodiment. The third light guide 113 is the same as the first light guide 111 and the second light guide 112 in which a two-step configuration of a first portion that receives light emitted from the third indicator LED 103 and guides received light, and a second portion that is connected to the first portion and exposes a part of a surface on the front surface 16 is made. Note that the third light guide 113 is composed to have the size, shape, and the like of the third indicator 53. In the embodiment, similarly to the second light guide 112 in FIG. 7, the third light guide 113 has a configuration in which the second portion is connected on the first portion (Z-direction positive side), and an end portion on the Y-direction positive side of the first portion is flush with an end portion on the Y-direction positive side of the second portion.

In the embodiment, the first indicator LED 101 and the third indicator LED 103 are provided on the LED substrate 109 common to the second indicator LED 102. Then, the first light shielding member 120 covers the entire LED substrate 109 including the first indicator LED 101, the second indicator LED 102, and the third indicator LED 103 with respect to the image detection unit 94. For this reason, the first light shielding member 120 shields the image detection unit 94 from the substrate (LED substrate 109) that controls the first indicator 51, the substrate (LED substrate 109) that controls the second indicator 52, and the substrate (LED substrate 109) that controls the third indicator 53. The first light shielding member 120 is, for example, a black sponge, tape, or the like. In this way, the first light shielding member 120 is provided, whereby it is possible to restrain light emitted from each indicator LED provided on the LED substrate 109 from leaking to the image detection unit 94. As in the embodiment, in a case where the image detection unit 94 is an indirect conversion type detector, and in a case where light leaks from the LED substrate 109 to the image detection unit 94, an artifact is generated in a radiographic image to be captured. For this reason, the first light shielding member 120 can restrain or reduce the artifact of the radiographic image.

The first indicator LED 101, the second indicator LED 102, and the third indicator LED 103 can be separate LED substrates. Alternatively, only one of the first indicator LED 101, the second indicator LED 102, and the third indicator LED 103 can be provided on a separate substrate. In the cases, the first light shielding member 120 may cover the respective substrates, on which the LEDs are mounted, to shield the image detection unit 94 from light emitted from the LEDs or may cover the whole of the respective substrates, on which the LEDs are mounted, to shield the image detection unit 94 from light emitted from the LEDs.

Although reduction in cost can be achieved in a case where a configuration is made in which the light emitter, such as an LED, of the indicator or the like is mounted on the control substrate 96 of the image detection unit 94, and light is guided from the light emitter on the control substrate 96, in the embodiment, as described above, the LED substrate 109 constituting the first indicator 51, the second indicator 52, and the third indicator 53 is formed of a substrate separated from the control substrate 96 of the image detection unit 94. The reason is more reliably for restraining light fogging (light leakage) to the image detection unit 94 by covering the entire LED substrate 109 with the first light shielding member 120. That is, the LED substrate 109 is formed of a substrate separate from the control substrate 96 of the image detection unit 94, and the first light shielding member 120 is provided to cover the LED substrate 109, whereby it is possible to more reliably restrain light fogging to the image detection unit 94. As a result, even though a plurality of light emitting indicators are provided on the front surface 16, the electronic cassette 10 can obtain an accurate radiographic image with no artifact or little artifact due to light fogging from the indicators.

The electronic cassette 10 further comprises a second light shielding member 121 that shields the first indicator 51 and the second indicator 52 from each other between the first indicator 51 and the second indicator 52 adjacent to each other. Similarly, the electronic cassette 10 further comprises a second light shielding member 122 that shields the second indicator 52 and the third indicator 53 from each other between the second indicator 52 and the third indicator 53 adjacent to each other.

The second light shielding members 121 and 122 are formed by the frame 92 (see FIG. 8). That is, the first light guide 111, the second light guide 112, and the third light guide 113 have a configuration in which each light guide is fitted to a groove portion formed by roughly cutting out a part of the frame 92 in the shape of the light guide. For this reason, a portion of the frame 92 remains between the first light guide 111 and the second light guide 112 constitutes the second light shielding member 121. A portion of the frame 92 that remains between the second light guide 112 and the third light guide 113 constitutes the second light shielding member 122. The second light shielding member 121 that is provided between the first light guide 111 and the second light guide 112 restrains light leakage between the first light guide 111 and the second light guide 112. As a result, in a case where the first indicator 51 is turned on, the second indicator 52 that is not turned on can be restrained from appearing to be turned on, and in a case where the second indicator 52 is turned on, the first indicator 51 that is not turned on can be restrained from appearing to be turned on. The second light shielding member 121 also restrains color mixing of the first indicator 51 and the second indicator 52. For this reason, the first indicator 51 and the second indicator 52 can be turned on correctly in predetermined display colors. Similarly, the second light shielding member 122 that is provided between the second light guide 112 and the third light guide 113 restrains light leakage between the second light guide 112 and the third light guide 113. As a result, the second light shielding member 122 can restrain erroneous recognition of turning-on and color mixing of the second indicator 52 and the third indicator 53.

In a case where the second light shielding members 121 and 122 are formed by the frame 92, the portions that are cut to fit the first light guide 111, the second light guide 112, and the third light guide 113 are minimized. For this reason, there is an advantage that the rigidity of the frame 92 can be maintained compared to a case where the portion related to the display unit 50 and the entire periphery are cut out and the first indicator 51, the second indicator 52, and the third indicator 53 are disposed.

In a portion where at least the first indicator 51, the second indicator 52, and the third indicator 53 are formed, the frame 92 has a two-step configuration of a first step portion 131 and a second step portion 132. The first step portion 131 is a portion that constitutes holes, to which the first portions (the first portion C1 of the second light guide 112, and the like in the FIG. 7) of the first light guide 111, the second light guide 112, and the third light guide 113 are fitted. The second step portion 132 is a portion that protrudes from the first step portion 131 toward the Z-direction positive side, and covers the side surfaces on the X-direction positive side and in the Y direction of the second portion C2 of the second light guide 112 and the second portion of the third light guide 113.

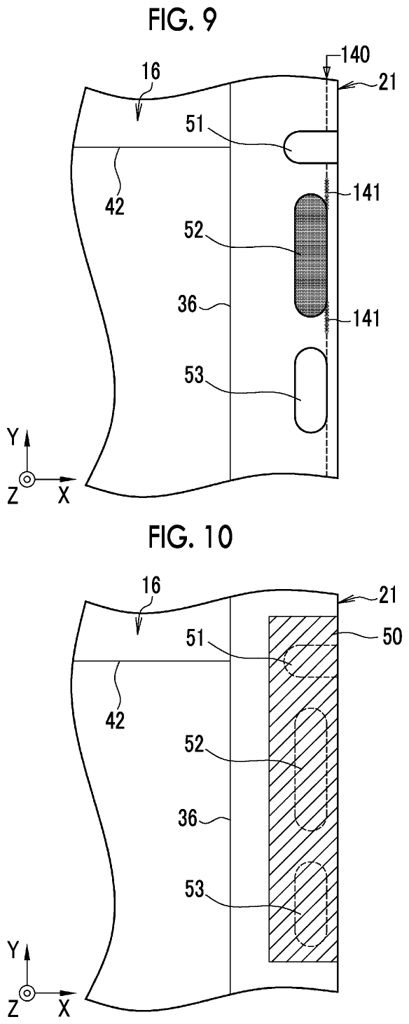

As shown in FIG. 9, in a case where the frame 92 does not have a two-step configuration of the first step portion 131 and the second step portion 132, and the second portions of the second light guide 112 and the third light guide 113 are exposed in the frame 92, and for example, in a case where the second indicator 52 is turned on, light leakage 141 occurs along a boundary 140 between the front plate 91 and the frame 92. Such light leakage 141 is likely to occur in a case where the end portion on the Y-direction positive side of the first portion C1 is flush with an end portion on the Y-direction positive side of the second portion C2. For this reason, the second step portion 132 is provided around at least the second light guide 112 and the third light guide 113 having the structure, such that and the boundary 140 between the frame 92 and the front plate 91 does not overlap a boundary between the frame 92 and each of the second light guide 112 and the third light guide 113. With this, light leakage 141 is restrained or reduced, and the second indicator 52 and the third indicator 53 can be turned on correctly in the shape exposed on the front surface 16.

In the above-described embodiment, although the second indicator 52 represents the ready state when turned on and represents the unready state when turned off, the second indicator 52 can further blink to represent another state of the electronic cassette 10. For example, the second indicator 52 can blink to represent a state in which the electronic cassette 10 or the system that uses the electronic cassette 10 is activated (during a preparation operation), or the like. In a case where the second indicator 52 represents a state of the electronic cassette 10 other than the ready state and the unready state by means of blinking, it is possible to increase the number of kinds of states of the electronic cassette 10 that can be reported on the front surface 16 without adding an indicator on the front surface 16 or in the display unit 50. Similarly, although the first indicator 51 is turned on to display the center of the imaging region and the identification information of the electronic cassette 10, the first indicator 51 can blink to display another state of the electronic cassette 10. Although the third indicator 53 is turned on to display the orientation of the image detection unit 94, the third indicator 53 can blink to display another state of the electronic cassette 10. In this way, in a case where the state of the electronic cassette 10 is displayed by means of blinking of the first indicator 51 or the third indicator 53, it is possible to increase the number of kinds of states of the electronic cassette 10 that can be reported on the front surface 16 without adding an indicator on the front surface 16 or in the display unit 50.

In the above-described embodiment, although the luminance of the second indicator 52 is made greater in comparison of the first indicator 51 and the third indicator 53, and the second indicator 52 is turned on the brightest, the second indicator 52 may be turned on relatively the brightest using a method other than increasing the luminance. That is, the comparison of the brightness of the first indicator 51, the second indicator 52, and the third indicator 53 can be performed using information other than luminance. Specifically, the luminous intensity, luminous flux (lumen), or the like of the second indicator 52 is made greater than the first indicator 51 and the third indicator 53, whereby the second indicator 52 can be turned on relatively the brightest. It is particularly preferable that the brightness of the first indicator 51, the second indicator 52, and the third indicator 53 is determined in view of not only the status relating to light emission of each indicator, such as luminance, luminous intensity, or luminous flux, but also spectral luminous efficiency (at least one of spectral luminous efficiency in a bright place or spectral luminous efficiency in a dark place). The reason is for allowing the radiological technician to recognize the second indicator 52 relatively the brightest in an actual use environment of the electronic cassette 10 in view of the display colors of the first indicator 51, the second indicator 52, and the third indicator 53. For example, it is preferable that a product of luminance, luminous intensity, luminous flux, or the like and spectral luminous efficiency (or an integrated value of the product for a wavelength) is made relatively the greatest, such that the second indicator 52 is turned on relatively the brightest.

In the above-described embodiment, although the display color of the second indicator 52 is set to an invariable fixed color, the display color of the second indicator 52 may be made changeable. In a case where the display color of the second indicator 52 is variable, for example, the second indicator 52 is turned on in the fixed color (green) of the above-described embodiment in turning-on during a normal operation, and the second indicator 52 is turned on in a color other than the fixed color of the above-described embodiment in turning-on when an error occurs. In this way, in a case where the display color of the second indicator 52 is made variable, a state of the electronic cassette 10 other than the ready state can be displayed on the front surface 16 of the electronic cassette 10 with the display color of the second indicator 52 or change in display color without adding an indicator in the display unit 50.

As in the above-described embodiment, the display units 50 on the respective sides of the electronic cassette 10 have symmetry that the first indicator 51, the second indicator 52, and the third indicator 53 are arranged in the same arrangement order as viewed from the direction of the center of the imaging region, and the arrangement order is roughly point symmetry (rotational symmetry). In the above-described embodiment, the first indicator 51, the second indicator 52, and the third indicator 53 are arranged in an arrangement order from the left when viewed from the imaging center (see FIG. 1 or the like). In this way, in the display units 50 that are provided on the respective sides of the electronic cassette 10, in a case where the arrangement order of the indicators is an arrangement order that is point symmetry, the LED substrate 109 that controls the first indicator 51, the second indicator 52, and the third indicator 53, and the like can be formed using the same parts in the respective display units 50. As a result, it is possible to reduce cost caused by providing a plurality of display units 50 in the electronic cassette 10.

In the above-described embodiment, as described above, although it is preferable that the first indicator 51, the second indicator 52, and the third indicator 53 are arranged in the arrangement order that is point symmetry, the arrangement order of the indicators may be a line-symmetric arrangement order in the X direction or the Y direction. In a case where the first indicator 51, the second indicator 52, and the third indicator 53 of each display unit 50 are arranged in the line-symmetric arrangement order, the arrangement order is the same in the two display units 50 among the display units 50 provided on the four sides of the electronic cassette 10. For this reason, since the LED substrates 109 and the like of the display units 50 can be formed using the same parts, it is possible to reduce cost compared to a case where the arrangement order of the indicators is different among the sides. Specifically, in the display units 50 on the upper side and the lower side, the first indicator 51, the second indicator 52, and the third indicator 53 can be arranged in this order from the left (X-direction negative side) along the X direction, and in the display units 50 on the right side and the left side, the first indicator 51, the second indicator 52, and the third indicator 53 can be arranged in this order from the top (Y-direction positive side) along the Y direction. In this case, since the arrangement order of the indicators is the same in the display units 50 on the upper side and the right side, and the arrangement order of the indicators is the same in the display units 50 on the lower side and the left side, it is possible to form the LED substrate 109 and the like in the display units 50 on the upper side and the right side using the same parts, and to form the LED substrate 109 and the like in the display unit 50 on the lower side and the left side using the same parts.

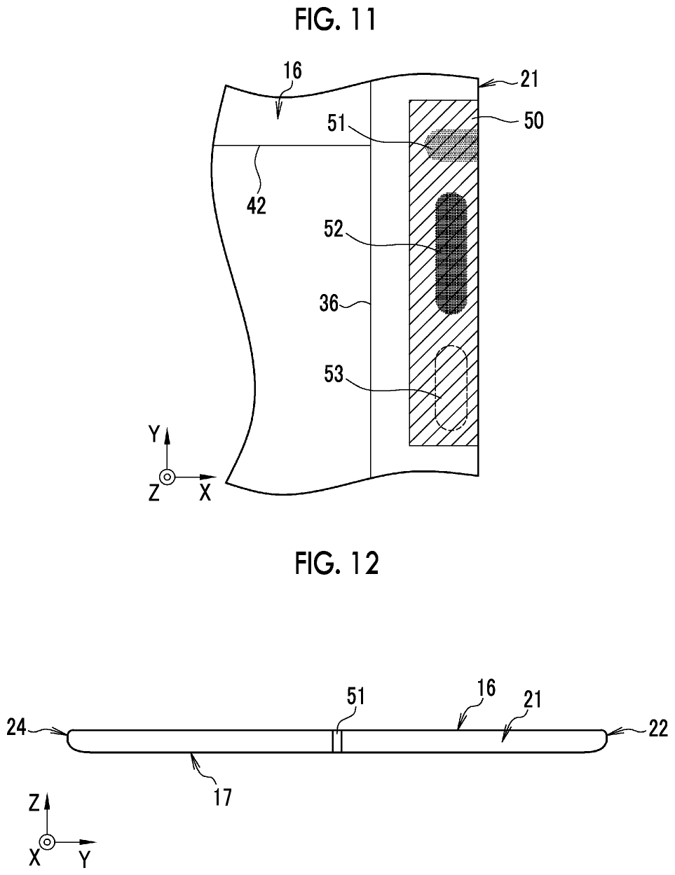

In the above-described embodiment, the display unit 50 is configured such that the first indicator 51, the second indicator 52, and the third indicator 53 are exposed directly on the front surface 16. However, as shown in FIG. 10, the display unit 50 can be configured such that the surfaces of the first indicator 51, the second indicator 52, and the third indicator 53 are hidden by, for example, the same black transparent plate as the front plate 91. In this case, the first indicator 51, the second indicator 52, and the third indicator 53 cannot be substantially viewed in a non-turning-on state. On the other hand, as shown in FIG. 11, for example, in a case where the first indicator 51 and the second indicator 52 are turned on, light is transmitted through the transparent plate, and the display of the first indicator 51 and the second indicator 52 that are turned on can be viewed. The same applies to a case where the third indicator 53 is turned on. In this way, in a case where the display unit 50 is constituted of the transparent place that hides the presence of the first indicator 51, the second indicator 52, and the third indicator 53 during non-turning-on, since each indicator appears as needed, it is possible to enhance the turning-on and the color of each indicator for the radiological technician. As a result, the radiological technician easily notices a content to be reported by each indicator without error.

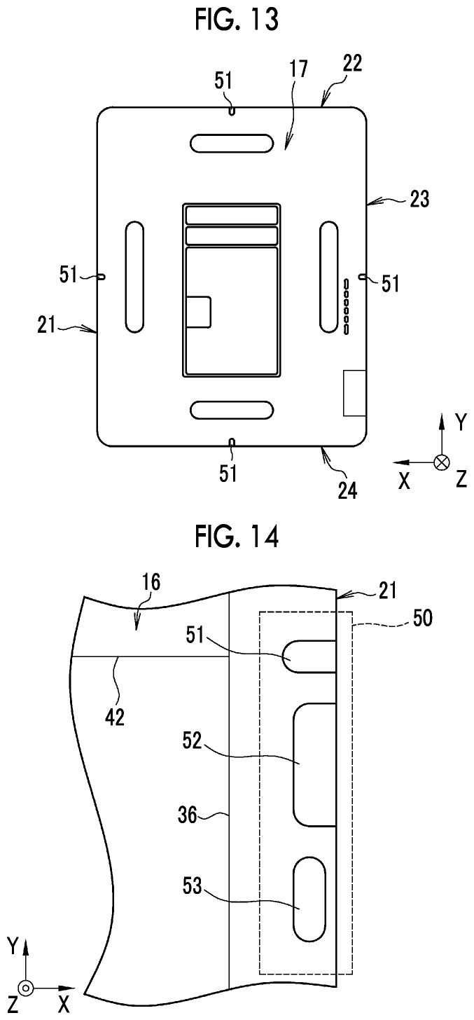

In the above-described embodiment, although the first indicators 51 are formed in a shape extended from the front surface 16 to the respective side surfaces 21 to 24, as shown in FIGS. 12 and 13, the first indicators 51 may have a shape extended to the rear surface 17. In this case, the identification color displayed by the first indicator 51 can also be ascertained from the rear surface 17 of the electronic cassette 10, and usability is improved.

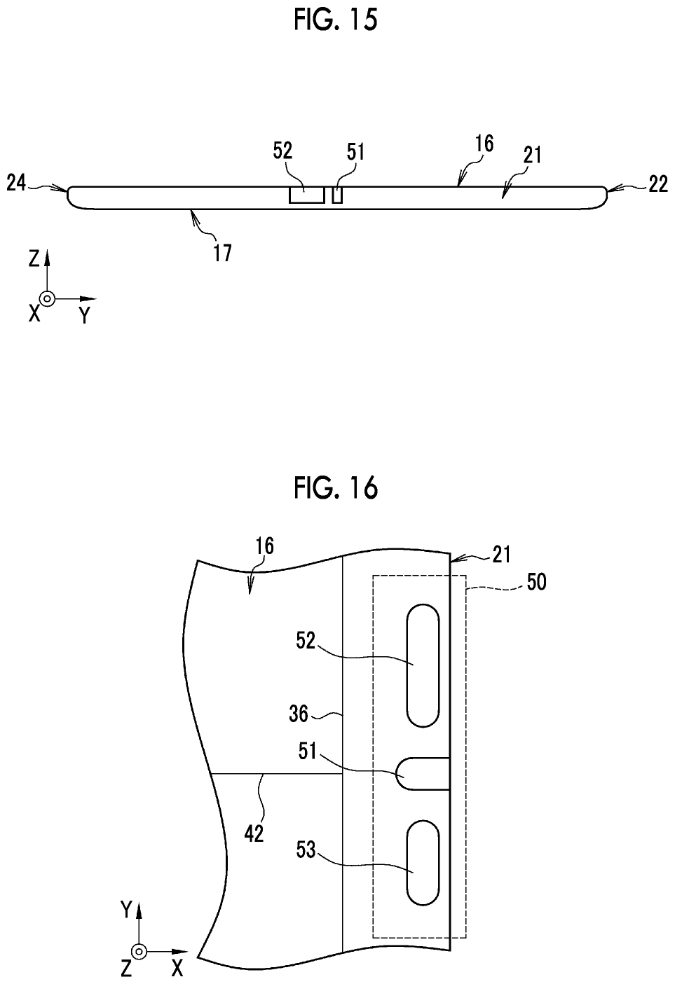

In the above-described embodiment, although the first indicators 51 are formed in a shape extended from the front surface 16 to the respective side surfaces 21 to 24, as shown in FIGS. 14 and 15, the second indicators 52 can be formed in a shape extended to the respective side surfaces 21 to 24. In this case, the turning-on of the second indicators 52 can be confirmed on the side surfaces 21 to 24. Similarly, the third indicators 53 can be formed in a shape extended to the respective side surfaces 21 to 24. The second indicators 52 and the third indicators 53 may be formed in a shape extended to the rear surface 17 like the first indicators 51 of FIGS. 12 and 13.

In the above-described embodiment, for example, although the indicators are arranged in the order of the first indicator 51, the second indicator 52, and the third indicator 53 from the Y-direction positive side on the right side, in regard to the arrangement of the indicators in the display unit 50, a relative arrangement of the indicators in the display unit 50 is discretionary excluding that the first indicator 51 is disposed on the extension of the crossline 41 or 42. For this reason, as shown in FIG. 16, for example, the indicators can be arranged in the order of the second indicator 52, the first indicator 51, and the third indicator 53 from the Y-direction positive side on the right side.

### EXPLANATION OF REFERENCES

10: electronic cassette

16: front surface

17: rear surface

21 to 24: side surface

31 to 34: marker

36 to 39: line

41, 42: crossline

50: display unit

51: first indicator

52: second indicator

53: third indicator

61 to 64: rubber members

71: center portion

72: first rear display unit

73: second rear display unit

74: battery pack

81, 82: operating unit

83 to 86: rear indicator

91: front plate

92: frame

93: rear plate

94: image detection unit

95: radiation shielding member

96: control substrate

101: first indicator LED

102: second indicator LED

103: third indicator LED

109: LED substrate

110: fixing member

111: first light guide

112: second light guide

113: third light guide

120: first light shielding member

121, 122: second light shielding member

131: first step portion

132: second step portion

140: boundary

141: light leakage

What is claimed is:

1. An electronic cassette comprising:
an image detection unit that detects a radiographic image of a subject using radiation transmitted through the subject;
a housing that houses the image detection unit and has a front surface directing toward the subject in a case of detecting the radiographic image, a rear surface facing the front surface, and side surfaces connecting the front surface and the rear surface;
a first indicator that is provided on the front surface and outside an imaging region of the image detection unit, and emits light to indicate a center of the imaging region; and
a second indicator that is provided on the front surface and outside the imaging region, and emits light to represent a possibility of detection of the radiographic image.

2. The electronic cassette according to claim 1,
wherein the first indicator and the second indicator are provided at a plurality of places of the housing.

3. The electronic cassette according to claim 2,
wherein the first indicator and the second indicator are provided on each of four sides of the front surface.

4. The electronic cassette according to claim 1,
wherein the second indicator is provided in a connection portion of the front surface and each of the side surfaces.

5. The electronic cassette according to claim 1,
wherein a display color of the first indicator and a display color of the second indicator are different.

6. The electronic cassette according to claim 5,
wherein the display color of the second indicator is green.

7. The electronic cassette according to claim 5,
wherein, in a case where another electronic cassette having the first indicator is usable, the first indicator has a variable display color, and emits light in a color different from the first indicator of the other electronic cassette.

8. The electronic cassette according to claim 1,
wherein, in a case where the second indicator and the first indicator are turned on, the second indicator is turned on brighter than the first indicator.

9. The electronic cassette according to claim 1,
wherein a display area of the second indicator on the front surface is greater than a display area of the first indicator on the front surface.

10. The electronic cassette according to claim 1,
wherein the first indicator has a long shape in a direction perpendicular to a side of the front surface, and the second indicator has a long shape in a direction parallel to the side of the front surface.

11. The electronic cassette according to claim 1,
wherein the first indicator has a shape extended to each of the side surfaces of the housing.

12. The electronic cassette according to claim 1,
wherein a substrate that controls the first indicator and a substrate that controls the second indicator are different from a control substrate of the image detection unit.

13. The electronic cassette according to claim 12, further comprising:
a first light shielding member that shields the image detection unit from the substrate that controls the first indicator and the substrate that controls the second indicator.

14. The electronic cassette according to claim 1, further comprising:
a second light shielding member that is provided between the first indicator and the second indicator to shield the first indicator and the second indicator from each other.

15. The electronic cassette according to claim 1, further comprising:
a third indicator that is provided on the front surface of the housing and outside the imaging region, and emits light to represent an orientation of the image detection unit.

16. The electronic cassette according to claim 15,
wherein the third indicator is provided on each of four sides of the front surface and is alternatively turned on.
